(19) United States
(12) Patent Application Publication
(10) Pub. No.: US 2026/0345705 A1
(43) Pub. Date: Nov. 5, 2026

(54) COMPOSITIONS FOR WOUND HEALING

(71) Applicant: Groupe Santé Devonian Inc., Montmagny (CA)

(72) Inventors: André P. BOULET, Montmagny (CA); Nathalie BOUCHER, Montmagny (CA)

(21) Appl. No.: 19/133,597

(22) PCT Filed: Nov. 2, 2023
(86) PCT No.: PCT/CA2023/051465

§ 371 Date: May 28, 2025

Related U.S. Application Data
(60) Provisional application No. 63/381,991, filed on Nov. 2, 2022.

(51) Int. Cl. A61K 36/21; A61P 17/02

(52) U.S. Cl. CPC A61K 36/21; A61P 17/02; A61K 2236/13; A61K 2236/15; A61K 2236/51; A61K 2236/53

(57) ABSTRACT

There is provided the use of a functional thylakoid extract, particularly in admixture with a physiologically acceptable carrier, in pharmaceutical application for wound healing. The thylakoid extract may be used to treat skin or mucosal wounds, such as open wounds or wounds caused by physical trauma. The thylakoid extract may be used to increase GM-CSF growth factor product or levels and/or G-CSF growth factor production or levels in the skin or mucosa of a subject following a wound (e.g., skin, mucosal, or corneal epithelial wound).

### FIELD

This invention relates to a composition comprising functional thylakoids, particularly in specific formulations that ensure the integrity, stability and functionality of the thylakoids (i.e., functional thylakoid extract), and methods of use for wound healing.

### BACKGROUND

WO 01/49305 discloses anti-oxidative compositions and method for their extraction. WO 03/04042 discloses their use in combination with other anti-inflammatory compounds. WO 2005/027944 discloses an oral formulation for the administration as anti-inflammatory compounds.

There is a need to develop new compositions and formulations for wound healing.

### SUMMARY

Therefore, there is provided a composition, comprising a functional thylakoid extract, or a pharmaceutical formulation thereof, and its use for wound healing.

Thus, there is also provided a composition, comprising a functional thylakoid extract, or a pharmaceutical formulation thereof, and its use in wound healing.

In a first aspect, there is provided a composition for wound healing in a subject, the composition comprising an effective amount of a functional thylakoid extract, particularly in admixture with a physiologically acceptable carrier.

In a second aspect, there is provided use of a functional thylakoid extract in the manufacture of a medication for wound healing in a subject.

In a further aspect, there is provided use of a functional thylakoid extract for wound healing in a subject.

In a further aspect, there is also provided a method for wound healing in a subject in need thereof, comprising topically administering to said subject an effective amount of a functional thylakoid extract, optionally in admixture with a physiologically acceptable carrier.

In a further aspect, there is also provided a topical composition comprising an effective amount of a photosynthetic cell extract, optionally in admixture with a physiologically acceptable carrier, for increasing GM-CSF growth factor production or levels and/or G-CSF growth factor production or levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

In a further aspect, there is also provided a topical composition for use in epithelial (e.g., skin, mucosa, or corneal epithelium) wound healing in a subject, comprising an effective amount of a photosynthetic cell extract, optionally in admixture with a physiologically acceptable carrier, wherein the photosynthetic cell extract is from spinach thylakoid and comprises an active thylakoid extract.

In a further aspect, there is also provided a use of a topical composition as defined herein for epithelial (e.g., skin, mucosa, or corneal epithelium) wound healing in a subject.

In a further aspect, there is also provided a use of a topical composition as defined herein for increasing GM-CSF growth factor levels and/or G-CSF growth factor levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

In a further aspect, there is also provided a method for epithelial (e.g., skin, mucosa, or corneal epithelium) wound healing in a subject in need thereof, said method comprising administering a topical composition as defined herein.

In a further aspect, there is also provided a method for increasing GM-CSF growth factor levels and/or G-CSF growth factor levels in the epithelial tissue (e.g., skin, mucosa, or corneal epithelium) of a subject following an epithelial wound, as compared to an untreated control, said method comprising administering a topical composition as defined herein.

Other objects, advantages and features of the present invention will become more apparent upon reading of the following non-restrictive description of preferred embodiments thereof, given by way of example only with reference to the accompanying drawings.

The contents of the documents cited in the present disclosure are incorporated by reference thereto.

### DETAILED DESCRIPTION

### Description of the Figures

FIG. 1 shows a flow diagram of the thylakoid extract manufacturing process.

FIG. 2 shows an HPLC chromatogram showing pigment profile of the thylakoid extract.

FIG. 3 shows a diagram showing the progression of phases of a wound healing in the skin following physical trauma. (Image from Vadivel and Brindha, 2017).

FIG. 4A shows the IFN-γ levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 4B shows the results from FIG. 4A plotted as percent change from untreated controls (i.e., cells that were not treated with the thylakoid extract).

FIG. 5A shows the IL-6 levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 5B shows the results from FIG. 5A plotted as percent change from untreated controls (i.e., cells that were not treated with the thylakoid extract).

FIG. 6A shows the IL-8 levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 6B shows the results from FIG. 6A plotted as percent change from untreated controls (i.e., cells that were not treated with the thylakoid extract).

FIG. 7A shows the IP-10 [CXCL-10] levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 7B shows the results from FIG. 7A plotted as percent change from untreated controls (i.e., cells that were not treated with the thylakoid extract).

FIG. 8A shows the MCP-1 levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 8B shows the results from FIG. 8A plotted as percent change from untreated controls (i.e., cells that were not treated with the thylakoid extract).

FIG. 9A shows the RANTES levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 9B shows the results from FIG. 9A plotted as percent change from untreated controls (i.e., cells that were not treated with the thylakoid extract).

FIG. 10A shows the TNF-α levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 10B shows the results from FIG. 10A plotted as percent change from untreated controls (i.e., cells that were not treated with the thylakoid extract).

FIG. 11A shows the Basic FGF levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 11B shows the results from FIG. 11A plotted as percent change from untreated controls (i.e., cells that were not treated with the thylakoid extract).

FIG. 12A shows the GM-CSF levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 12B shows the results from FIG. 12A plotted as percent change from untreated controls (i.e., cells that were not treated with the thylakoid extract).

FIG. 13A shows the G-CSF levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 13B shows the results from FIG. 13A plotted as percent change from untreated controls (i.e., cells that were not treated with the thylakoid extract).

FIG. 14A shows the elastin levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 14B shows the results from FIG. 14A plotted as percent change from untreated controls (i.e., cells that were not treated with the thylakoid extract).

FIG. 15A shows the pro-collagen levels during recovery from mechanical wound from the in vitro wound healing assay whereby human dermal fibroblasts were treated with the thylakoid extract (at 0.02 mg/L, 0.2 mg/L, or 2 mg/L) either before or after the mechanical wound under untreated conditions (i.e., direct effect). Statistical significance is assessed below. FIG. 15B shows the results from FIG. 15A plotted as percent change from untreated controls (i.e., cells that were not treated with the thylakoid extract).

### DEFINITIONS

The term “about” as used herein refers to a margin of + or −10% of the number indicated. For the sake of precision, the term about when used in conjunction with, for example: about 90% means 90%+/−9% i.e., from 81% to 99%. Alternatively, the term about refer to + or −5% of the number indicated, where for example: 90% means 90%+/−4.5% i.e., from 86.5% to 94.5%. Alternatively, the term about can also refer to + or −1, 2, 3, 4, 5, 6, 7, 8, 9 or 10% of the number indicated. Alternatively, the term about can also refer to the normal experimental variation of a given apparatus.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The terms: “thylakoid”, “thylakoid extract”, “functional thylakoid extract” or “functional thylakoid” or “active thylakoids/extract” as used herein, means purified functional photosynthetic pigments in a thylakoid membrane environment (i.e., in an integral native state such that they can still be active or activated), particularly their original thylakoid environment. More particularly, these terms refer to functional thylakoid membranes as extracted by the process herein described and/or by the procedure disclosed in Bissonnette et al. (2004), WO01/49305, WO2018081903A1, WO2011127559A1, or WO2017075698A1, which are all hereby incorporated herein by reference.

The term: “wound healing” as used herein comprises accelerating wound healing (e.g., skin, mucosa or corneal epithelium), reducing scarring and/or increasing tensile strength of the skin barrier in a subject relative to an untreated subject. In accordance with a particular aspect, the term “would healing” refers to a measurable or observable reduction of the wound, in the treated sample receiving treatment relative to an untreated control sample set.

Particularly, in connection with an aspect of the present thylakoid extract, the functional quality of the molecular complex can be measured by fluorescence based on its capacity to react to light and dissipate its energy (Fᵥ/Fₘ ratio), as is well known in the art and/or described in Maxwell (2000). In one aspect, the (Fᵥ/Fₘ ratio) of the functional thylakoid extract is at least 0.7.

### DETAILED DESCRIPTION OF PARTICULAR EMBODIMENTS

### Composition

In accordance with a particular aspect, the invention describes a composition for wound healing in a subject, comprising an effective amount of an active thylakoid extract, particularly in admixture with a physiologically acceptable carrier. Particularly, the thylakoid extract is a spinach thylakoid extract, and more particularly extracted from spinach leaves.

Particularly the composition comprises purified functional photosynthetic pigments in a thylakoid membrane environment. Still particularly, the extract is quiescent and can be activated photosynthetically. More particularly, the extract is stabilized in its fundamental state (i.e. stable) by being devoid of any electron donor (such as water). The extract can also be present in aqueous form.

Most particularly, the composition is defined as a raw organic spinach, active thylakoid extract, wherein the ratio chlorophyll a to total pigment or to total HPLC content is at least 0.4, particularly at least 0.5, more particularly at least 0.6.

In particular, the pigment comprised in the thylakoid extract is selected from the group consisting of chlorophyll a, chlorophyll b, and carotenoids. More particularly, the pigment comprised in the thylakoid extract is selected from the group consisting of: chlorophyll a, chlorophyll b, lutein, and optionally, β-carotene and/or pheophytin. Still, most particularly, the pigment comprised in the thylakoid extract consists essentially of: chlorophyll a (more than 40%), followed by chlorophyll b (about 10-15%), lutein (about 10% or less), β-carotene (about 3%) and pheophytin (less than 1%). In one aspect, the thylakoid extract has an HPLC profile substantially corresponding to FIG. 2. In one aspect, the thylakoid extract comprises chlorophyll a (about 62.5%), chlorophyll b (about 13.1%), lutein (about 9.4%), β-carotene (about 2.98%) and pheophytin (about 0.45%). In one aspect, the average ratio of chlorophyll a to total peak area response should not be less than 0.40 (with respect to all of the peaks). In one aspect, the thylakoid extract comprises chlorophyll a (about 62.5%), chlorophyll b (about 13.1%), lutein (about 9.4%), β-carotene (about 2.98%) and pheophytin (about 0.45%). In one aspect, the average ratio of chlorophyll a to total peak area response should not be less than 0.40. In one aspect, the thylakoid extract comprises no more than 10% w/w of water.

Particularly, the stabilized extract is in solid form, more as particularly, as a powder. Still, more particularly, the extract is in powder form with at least 25 mg pigments per gram of powder.

The composition according to the description can be prepared in and embodied in all pharmaceutical forms normally used for topical application. Furthermore, the composition may comprise the usual additives in the dermatological fields, such as fats, emulsifiers and co-emulsifiers, hydrophilic or lipophilic gelling agents, hydrophilic or lipophilic active ingredients, preservatives, antioxidants, solvents, fragrances, fillers, hydrophilic and lipophilic filters, dyestuffs, neutralizers, pro-penetrating agents and polymers.

The extract can be formulated in a liquid composition (a non-lyophilised extract), a lyophilized extract reconstituted in water, physiological saline or any other solution compatible with topical administration, in propylene glycol, or in a solid composition.

### Uses and Method

In accordance with a particular aspect, there is provided use of a thylakoid extract comprising purified functional photosynthetic pigments in a thylakoid membrane environment in the making of a medication for wound healing in a subject. Alternatively, there is provided use of a thylakoid extract comprising purified functional photosynthetic pigments in a thylakoid membrane environment for wound healing in a subject.

In accordance with a particular aspect, the thylakoid extract is for topical application/administration. Topical application/administration refers to administration/application to the epithelium including without limitation, skin, mucous membrane, corneal epithelium or the like. In accordance with a particular aspect, the thylakoid extract is for dermal topical application/administration to the skin.

In accordance with a particular aspect, the wound is an epithelial wound. In accordance with a particular aspect, the wound is a skin wound. In one aspect, the wound is a mechanical open wound caused by a mechanical force or a wound caused by physical trauma. In a further aspect, the wound comprises skin ulcer (e.g., neuropathic ulcer an ischemic ulcer or a neuroischemic ulcer), an infectious wound, an ischemic wound, a laceration, a surgical wound, a stab, a cut, a puncture, a skin wound from radiation poisoning, or a combination thereof. In a further aspect, the wound comprises skin ulcer (e.g., neuropathic ulcer an ischemic ulcer or a neuroischemic ulcer), an infectious wound, an ischemic wound, a surgical wound, a skin wound from radiation poisoning, or a combination thereof. In some aspects, the wound penetrates to the skin. In some aspects, the wound penetrates to the dermis. In some aspects, the wound penetrates to the subcutaneous hypodermis. In some aspects, the wound penetrates to the subcutaneous tissue. In some aspects, the wound penetrates to the muscle. In some aspects, the thylakoid extract is used to treat skin wounds in combination with other known treatments used to treat skin wounds (e.g., stitches, glue, staples, tape, bandages, antibiotics).

In some aspects, the thylakoid extract defined herein is used to treat mucosal wounds (e.g., any organ or tissue such as but not limited to nasal, oral, lung, anal, ear, genital, esophageal, bronchial, gastric, uterine, bladder, intestinal mucosa). For example, mucosal wounds may be caused by surgery (e.g., incisions) or by lacerations. In some aspects, the thylakoid extract defined herein is used during surgery.

In accordance with a particular aspect, the wound is not caused by UV A and/or B exposure or by autoimmune disorders or conditions such as eczema.

In accordance with a particular aspect, the treatment with the composition increases proinflammatory cytokine (e.g., IFN-γ, IL-6, IL-8, IP-10 [CXCL-10], MCP-1, RANTES, and/or TNF-α) production or levels (e.g., in any layer of the skin or mucosa), as compared to an untreated control.

In accordance with a particular aspect, the treatment with the composition increases collagen and/or pro-collagen production or levels (e.g., in any layer of the skin or mucosa), as compared to an untreated control.

In accordance with a particular aspect, the treatment with the composition increases elastin production or levels (e.g., in any layer of the skin or mucosa), as compared to an untreated control.

In accordance with a particular aspect, the treatment with the composition increases basic FGF, G-CSF and/or GM-CSF production or levels (e.g., in any layer of the skin or mucosa), as compared to an untreated control.

In some aspects, a colloidal effect by the thylakoid extract may be observed. For example, an increase in proinflammatory cytokine, growth factor, collagen, elastin and/or pro-collagen levels may not be as pronounced under certain concentrations (e.g., low and high concentrations) but may be observed at other concentrations (e.g., between low and high concentrations. In some aspects, a bell-shaped curve dose response is observed. This may be due to the downregulation or upregulation of certain cell surface receptors at different thylakoid extract concentrations.

In accordance with a particular embodiment, there is provided a method for wound healing in a subject in need thereof, comprising administering to the subject an effective amount of a thylakoid extract comprising purified functional photosynthetic pigments in a thylakoid membrane environment, particularly in admixture with a physiologically acceptable carrier.

In particular, the present use and method may be indicated for the treatment of mammalian subjects, particularly pets or human, more particularly cats, dogs, horses, or human, most particularly humans.

### Formulation

In accordance with a particular aspect, there is provided the use or the method of treatment as defined herein, wherein the composition is formulated for application to an epithelial tissue. As used herein, the term “epithelial tissue” comprises any epithelial tissue including, without limitation, skin, mucous membrane, corneal epithelium or the like.

### Dosage

As used herein, the terms “effective amount” means a dose sufficient to induce a accelerate wound healing and/or reduce the scaring and/or increasing tensile strength of the skin barrier.

In accordance with a particular embodiment, the extract is provided at a dosage between about 0.01% to about 0.2%, about 0.01% to about 0.1%, about 0.05% to 0.15%, about 0.075% to 0.125%, or about 0.9% to 0.11%.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### EMBODIMENTS

The following embodiments are provided:

Embodiment 1. A topical composition comprising an effective amount of a photosynthetic cell extract, optionally in admixture with a physiologically acceptable carrier, for increasing GM-CSF growth factor production or levels and/or G-CSF growth factor production or levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

Embodiment 2. A topical composition for use in epithelial (e.g., skin, mucosa, or corneal epithelium) wound healing in a subject, comprising an effective amount of a photosynthetic cell extract, optionally in admixture with a physiologically acceptable carrier, wherein the photosynthetic cell extract is from spinach thylakoid and comprises an active thylakoid extract.

Embodiment 3. The topical composition for use of any of the preceding embodiments, wherein the extract is present in an amount from about 0.010% to about 0.2% based upon the total weight of the composition.

Embodiment 4. The topical composition for use of any of the preceding embodiments, wherein the extract is for administration before the wound is present.

Embodiment 5. The topical composition for use of any of the preceding embodiments, wherein the extract is for administration after the wound is present.

Embodiment 6. The topical composition of any one of the preceding embodiments, wherein the carrier is selected from the group consisting of fats, hydrophilic or lipophilic gelling agents, solvents, and fillers.

Embodiment 7. The topical composition of any one of the preceding embodiments wherein the extract is non-lyophilized.

Embodiment 8. The topical composition of any one of the preceding embodiments, in the form of a solution, a cream, an ointment, an ointment, a foam, a spray, a transdermal patch, or a gel.

Embodiment 9. The topical composition of any one of the preceding embodiments, wherein the extract is a lyophilized extract reconstituted in water, in physiological saline or any other solution compatible with topical administration, in propylene glycol, or in a solid composition.

Embodiment 10. The topical composition of any one of the preceding embodiments, wherein the photosynthetic cell extract is from spinach thylakoid and comprises functional thylakoids.

Embodiment 11. The topical composition of any one of the preceding embodiments, wherein the active thylakoid extract comprises purified functional photosynthetic pigments in their thylakoid membrane environment.

Embodiment 12. The topical composition of any one of the preceding embodiments, wherein the wound is an open mechanical wound.

Embodiment 13. The topical composition of any one of the preceding embodiments, wherein the wound comprises skin ulcer (e.g., neuropathic ulcer an ischemic ulcer or a neuroischemic ulcer), an infectious wound, an ischemic wound, a surgical wound, a skin wound from radiation poisoning, or a combination thereof.

Embodiment 14. The topical composition of any one of the preceding embodiments, wherein the wound is not caused by UVA and/or UVB exposure or by autoimmune disorders or conditions such as eczema.

Embodiment 15. The topical composition of any one of the preceding embodiments, wherein treatment with the composition increases collagen production or levels in the epithelial tissue, as compared to an untreated control.

Embodiment 16. The topical composition of any one of the preceding embodiments, wherein treatment with the composition increases elastin production or levels in the epithelial tissue, as compared to an untreated control.

Embodiment 17. The topical composition of any one of the preceding embodiments, wherein treatment with the composition increases pro-collagen, collagen and/or elastin production or levels in the epithelial tissue, as compared to an untreated control.

Embodiment 18. The topical composition of any one of the preceding embodiments, wherein treatment with the composition increases basic FGF, G-CSF, and/or GM-CSF production or levels in the epithelial tissue, as compared to an untreated control.

Embodiment 19. The topical composition of any one of the preceding embodiments, wherein treatment with the composition increases proinflammatory cytokine (e.g., IFN-γ, IL-6, IL-8, IP-10 [CXCL-10], MCP-1, RANTES, and/or TNF-α) production or levels in the epithelial tissue, as compared to an untreated control.

Embodiment 20. Use of a topical composition as defined in any one of the preceding embodiments for epithelial (e.g., skin, mucosa, or corneal epithelium) wound healing in a subject.

Embodiment 21. Use of a topical composition as defined in any one of the preceding embodiments for increasing GM-CSF growth factor levels and/or G-CSF growth factor levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

Embodiment 22. A method for epithelial (e.g., skin, mucosa, or corneal epithelium) wound healing in a subject in need thereof, said method comprising administering a topical composition as defined in any one of the preceding embodiments.

Embodiment 23. A method for increasing GM-CSF growth factor levels and/or G-CSF growth factor levels in the epithelial tissue (e.g., skin, mucosa, or corneal epithelium) of a subject following an epithelial wound, as compared to an untreated control, said method comprising administering a topical composition as defined in any one of the preceding embodiments.

### EXAMPLES

### Example 1—Preparation of Thylakoid Extract

The thylakoid extract originates from the mesophyll tissue of baby spinach (Spinacia oleracea L.) leaves, which is rich in chloroplasts. The inner membranes of the chloroplasts, organized in structures known as thylakoids, are extracted from baby spinach, concentrated and stabilized into a solid powder form. The major constituents of thylakoid membranes are pigments, proteins and lipids. Characteristics of thylakoid extract are shown in Table 1.

TABLE 1
Thylakoid Extract Characteristics

| | |
|---|---|
| Physical appearance: | Dark green powder |
| Solubility in water: | Insoluble |
| Solubility in alkaline medium (pH 10.6) | 0.5 mg/mL |
| Solubility in acidic medium (pH 1.0) | 0.3 mg/mL |

### Manufacturing Process

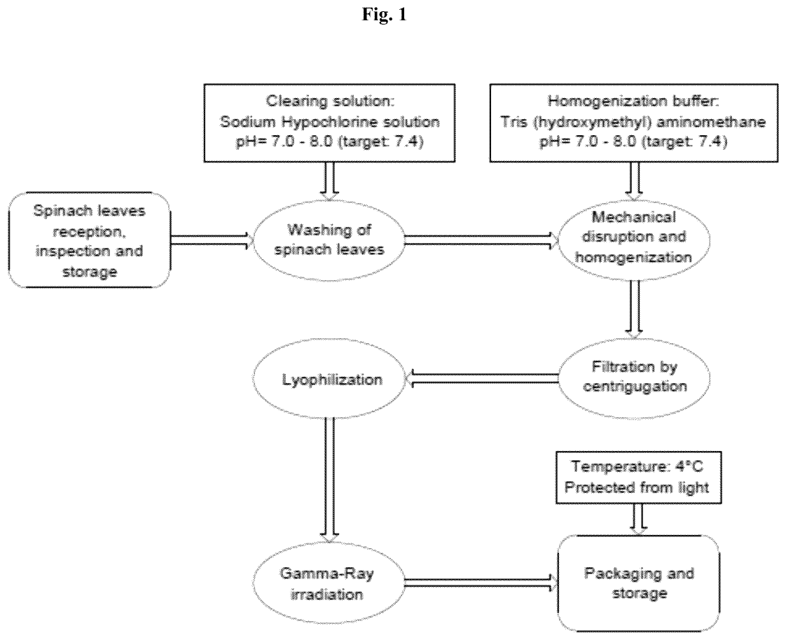

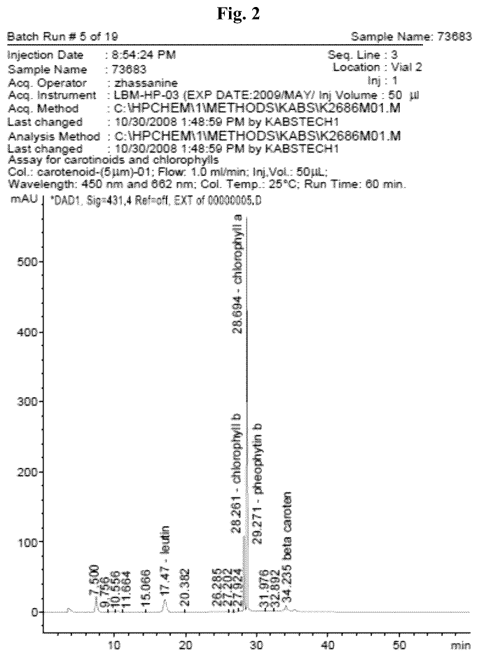

The manufacturing process for the thylakoid extract is presented schematically in the flow diagram of FIG. 1.

The processing steps are executed with minimum light exposure and under cool conditions to preserve a maximal activity of the photosynthetic pigments. The steps are carried out in the following order: inspection of spinach leaves and washing with a sodium hypochlorite solution; mechanical disruption and homogenization; filtration by centrifugation; lyophilisation; and gamma-ray irradiation.

Inspection of spinach leaves and washing with a sodium hypochlorite solution. After visual inspection is performed to verify dimensional and identity attributes (e.g., leaves are green without discoloured zones or yellowish pecks (chlorose)), spinach leaves are first washed at a fixed solution-to-leaves ratio (44 kg: 5.4 kg) on a mass basis, with a sodium hypochlorite solution adjusted to a pH between 7.0 and 8.0 (target pH: 7.4) to reduce the microbial flora naturally found on the leaves of fresh produce.

Mechanical disruption and homogenization. After draining the excess sodium hypochlorite solution, leaves are transferred into a mechanical cutter/mixer along with a fixed volume of Tris (hydroxymethyl) aminomethane buffer solution at pH between 7.0 and 8.0 (target pH: 7.4) at a fixed solution-to-leaves ratio (5.4 kg: 3.7 kg) on a mass basis. This step is used to cut and homogenize the leaves into a coarse suspension while freeing up fragments of the thylakoid membranes originating from chloroplasts.

Filtration by centrifugation. The suspension is then filtered in a basket centrifuge. The centrifugation is performed at a target speed of 3100 rpm (range: 2800-3200 rpm). This step allows the removal of fibres, debris and coarse material which are retained on a screen, yielding a by-product cake to be discarded. The thylakoid extract, the active ingredient, is found in the centrifugate and is collected and kept at a temperature below 10 °C for further processing.

Lyophilisation. The material is then distributed over shallow stainless-steel plates and allowed to freeze in darkness at a temperature≤−30 °C for a period of at least 2 hours. The plates kept at a target temperature of 10 °C are then transferred into a lyophilizer and the product is lyophilized.

Gamma-ray irradiation. A terminal gamma-ray irradiation step is carried out. After irradiation, the thylakoid extract is transferred into jars fitted with a tight screw cap.

The material can be subjected to filtration after centrifugation step. For example, size exclusion chromatography can be carried out to separate the most active fractions by molecular weight. Particularly, the crude thylakoids can be further purified on Sephadex G-100 filtration and the fractions corresponding to a ratio of Fᵥ/Fₘ of at least 0.7 recovered.

### Pigment Composition and Other Characteristics

Spinach contains natural antioxidants (e.g., flavonoids) and photosynthetic pigments (chlorophylls and carotenoids). The inner membranes of the chloroplasts are organized in structures known as thylakoids. The major constituents of thylakoid membranes are pigments, proteins and lipids.

The thylakoid extract originates from the mesophyll tissue of spinach leaves which are rich in chloroplasts. To date, the following pigments have been identified in the thylakoid extract using HPLC analysis: lutein, chlorophyll b, chlorophyll a, pheophytin and β-carotene. A typical chromatogram showing the pigment profile of the thylakoid extract, in area %, is presented in FIG. 2. This analysis shows that the major constituent of the thylakoid extract is chlorophyll a (62.5%), followed by chlorophyll b (13.1%), lutein (9.4%), β-carotene (2.98%) and pheophytin (0.45%).

Preferably, raw baby spinach was obtained from a grower certified as per the National Organic Standards of the United States Department of Agriculture (USDA) to minimize risks of presence of potential chemical residues from fertilizers or pesticides in the thylakoid extract.

### Justification of Specification

The thylakoid extract is characterized by its pigment content expressed in milligram of pigment per gram of powdered extract. Based on process capabilities and allowing for seasonal variability in the herbal starting material, a specification of not less than 25 mg pigment/g extract was set. Based on stability data, a limit of 80% of the initial pigment content was set for shelf-life.

Pigment profile also allows identification of the various pigments present in the thylakoid extract and their ratios in area percent. Given the profile determined in batches, it was established that chlorophyll a, chlorophyll b, lutein and β-carotene should be present and that the average ratio of chlorophyll a to total peak area response should not be less than 0.40.

Since water is used as extraction solvent in the manufacturing process, a test to determine water content in the thylakoid extract has been included. A specification of not more than 10% w/w of water was set to control moisture.

### Stability

Batches of the thylakoid extract medicinal product, packaged in ajar with a tight screw cap, are placed on stability under the following conditions: 5 °C±3 °C (current recommended storage condition). The data available indicates that the thylakoid extract is stable after storage for at least 18 months under refrigerated conditions.

### Example 2—Methods

### Wound Healing In-Vitro Assay: Mechanical Wound Model—Scratch Recovery on Dermal Fibroblast Cultures

The scratch recovery on dermal fibroblast cultures is the gold-standard in vitro assay to mimic certain aspects of wound healing, and as such it is also relevant for normal ongoing remodeling to protect the integrity of the skin barrier. Briefly, human primary dermal fibroblasts from a healthy adult donor are used for the testing. The fibroblasts are cultured in flat-bottomed 24-well plates until the fibroblasts had formed monolayers on the bottom of each well. In order to test whether applying the thylakoid extract before the wounding, some wells are pre-treated with the thylakoid extract prior to performing the mechanical wound. These same wells are also treated with the thylakoid extract after the wound is made. Using a sterile tool, a scratch is made across each well as a model for a mechanical wound. The culture medium is removed, and fresh medium added, to help remove any clumps of cells that may have formed during the wounding process. This helped reduce the amount of freely flowing cells that might settle into the wound area and confound the analysis of wound repair by cell migration. The cells are then immediately exposed to the thylakoid extract. Concentrations of the thylakoid extract used in the assay were selected based on cell viability assays conducted on human PBMCs and human dermal fibroblasts using an MTT assay.

The culture plates are allowed to rest for an hour, so that any clumps of loose cells that may have settled in the scratch zone can be documented, and not affect the final scores of scratch recovery. The cultures are examined after 16-24 hours, to monitor scratch recovery for product-treated cells, compared to control cell cultures. Culture supernatants are collected at 24 hours from both wounded and non-wounded cultures and banked frozen. Culture supernatants are then tested for levels or secreted pro-collagen (Type I) and elastin, using commercial ELISA assays. Cytokines of culture supernatants are tested in duplicate for a broad panel of pro and anti-inflammatory cytokines, anti-viral peptides, and regenerative growth factors, using a Luminex™ magnetic bead array and the MagPix™ multiplexing system.

### Example 3—Effect of Thylakoid Extract on Wound Healing

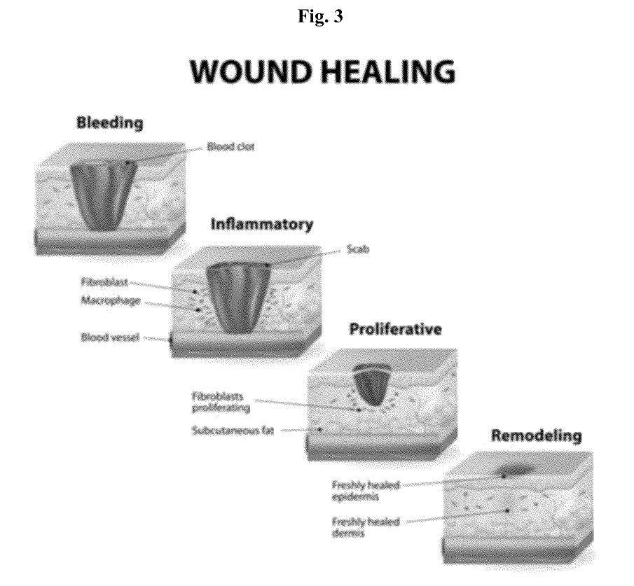

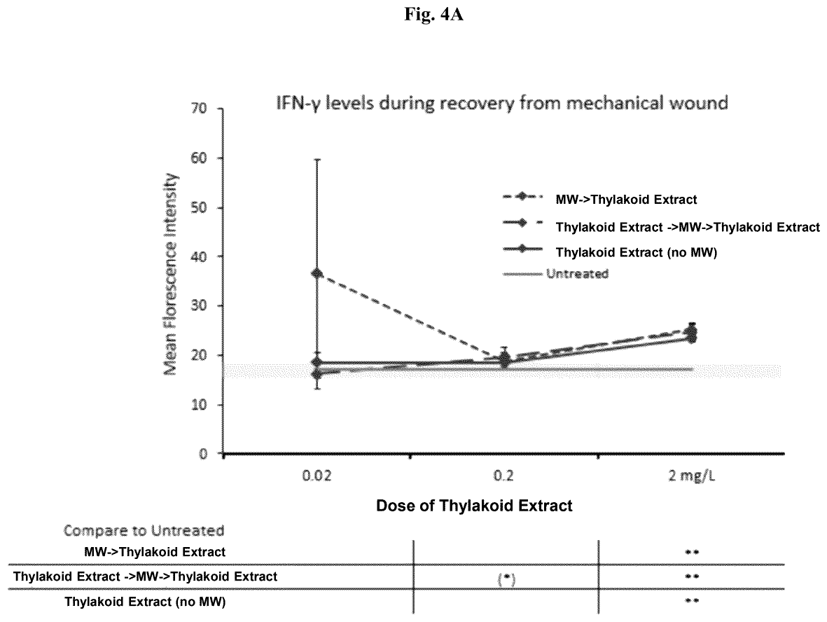

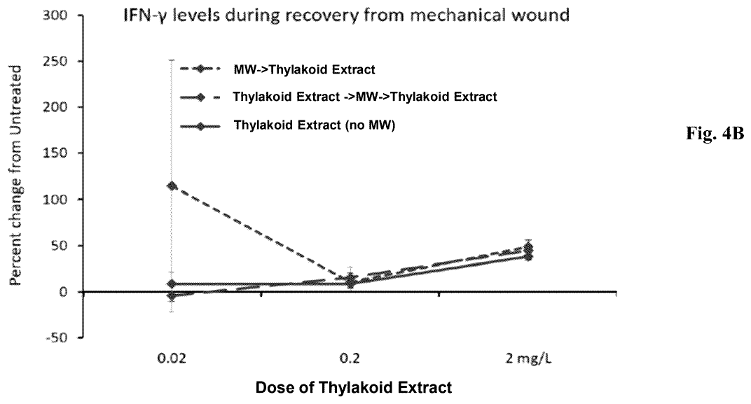

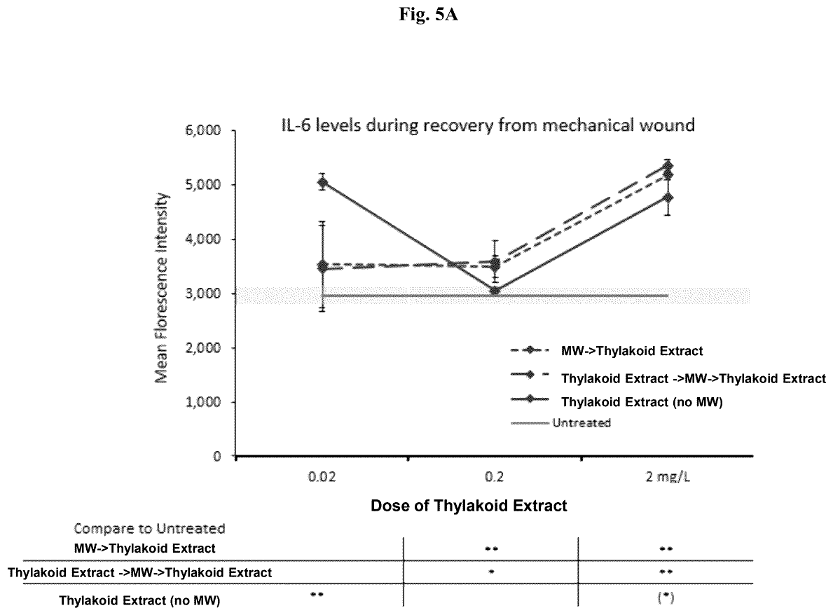

The effect of the thylakoid extract produced (as described in Example 1) in the in vitro wound healing assay (as described in Example 2) was assessed. As part of normal wound healing, the following phases comprise: an inflammatory phase, a proliferative phase, and a remodelling phase (FIG. 3). The freshly wounded skin will rapidly enter into an inflammatory phase, designed to signal the acute damage to the skin and underlying tissue, and trigger recruitment of fibroblasts and macrophages to assist in the repair process. The fibroblasts enter into a proliferative growth phase, in part supported by various growth factors. Finally, cell migration is part of remodeling, as the skin regains its tactile strength.

### Inflammatory Phase

FIG. 4 shows the summary of the proinflammatory cytokine multiplex from the in vitro wound healing assay whereby human dermal fibroblasts were treated with the thylakoid extract (0.02 mg/L, 0.2 mg/L, or 2 mg/L) either before or after the mechanical wound. Results are plotted as proinflammatory cytokine (IFN-γ, IL-6, IL-8, IP-10 [CXCL-10], MCP-1, RANTES, or TNF-α) percent change increase or decrease from untreated controls (i.e., cells that were not treated with the thylakoid extract).

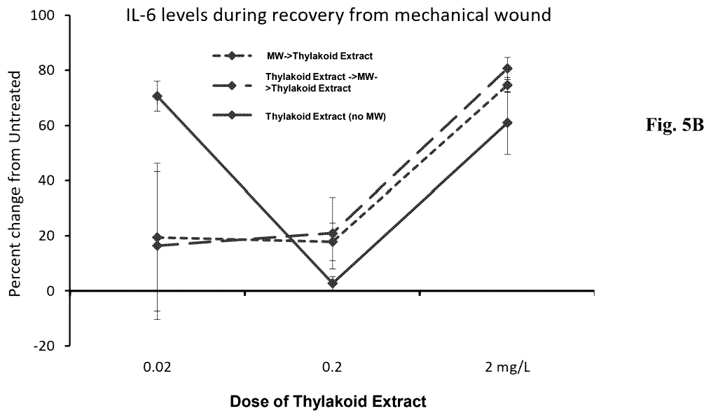

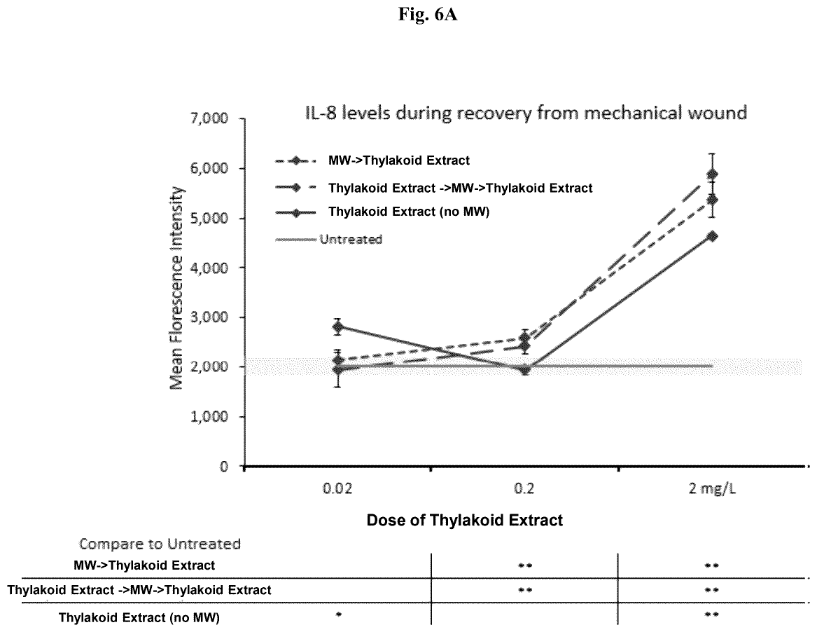

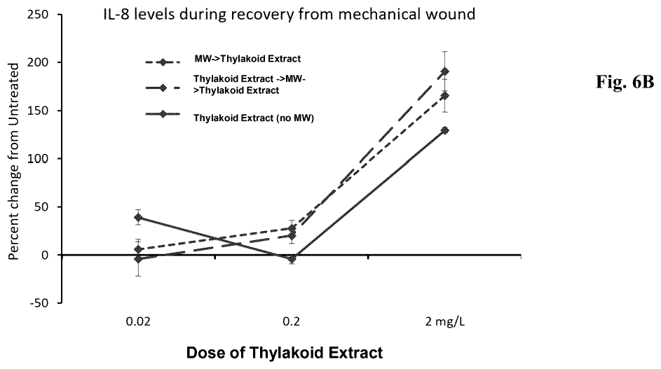

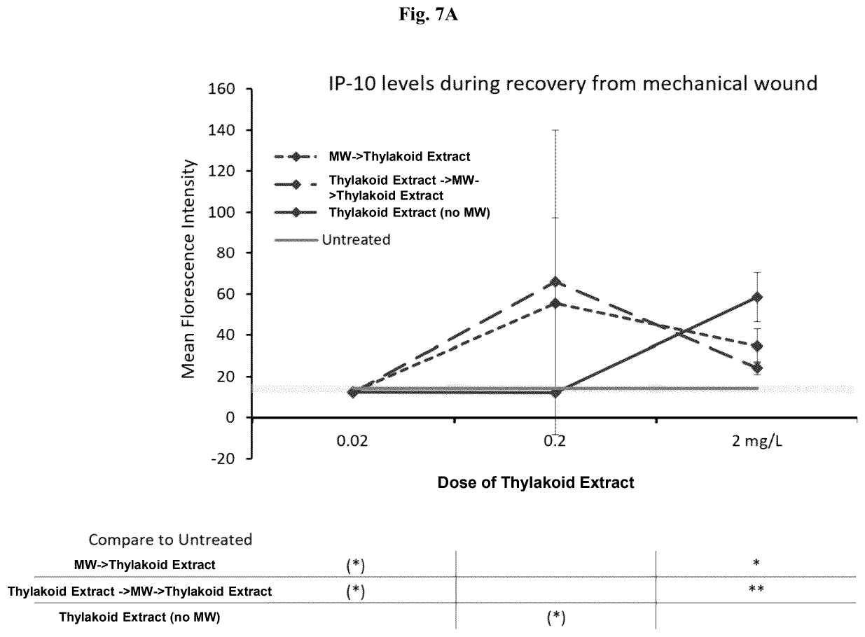

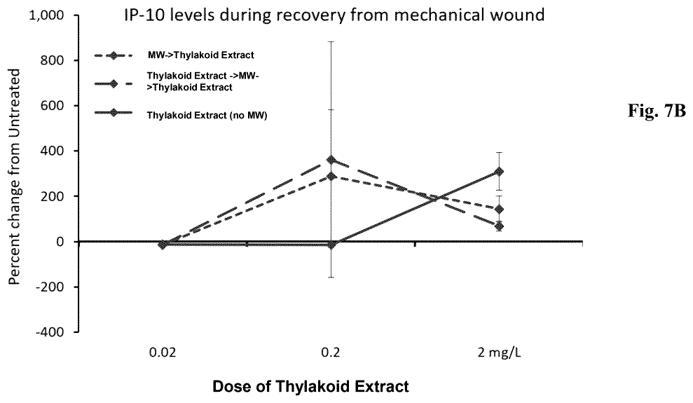

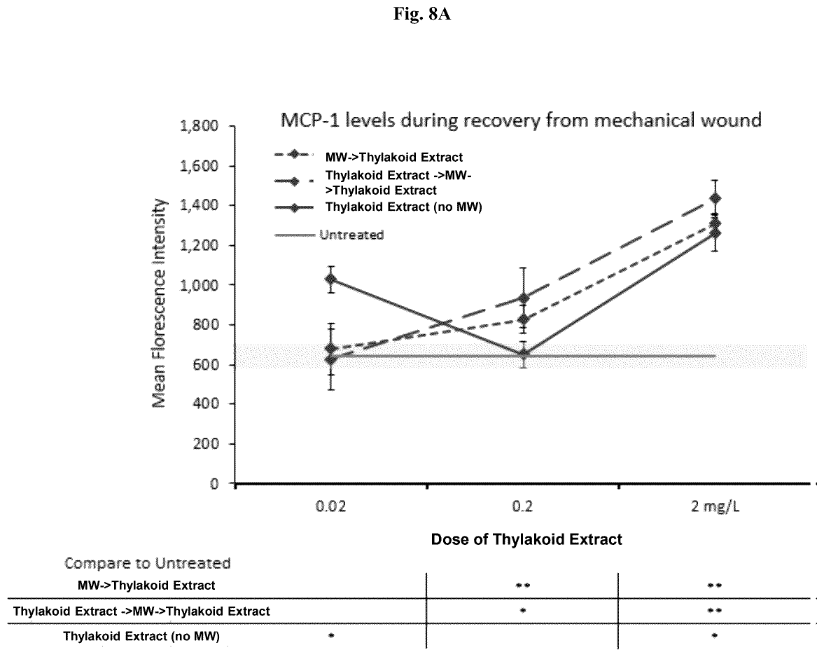

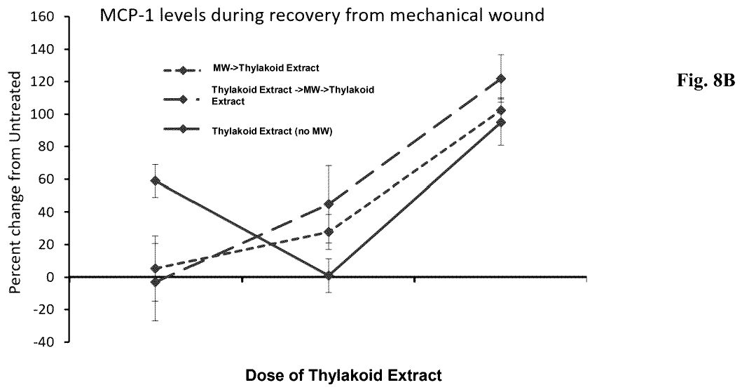

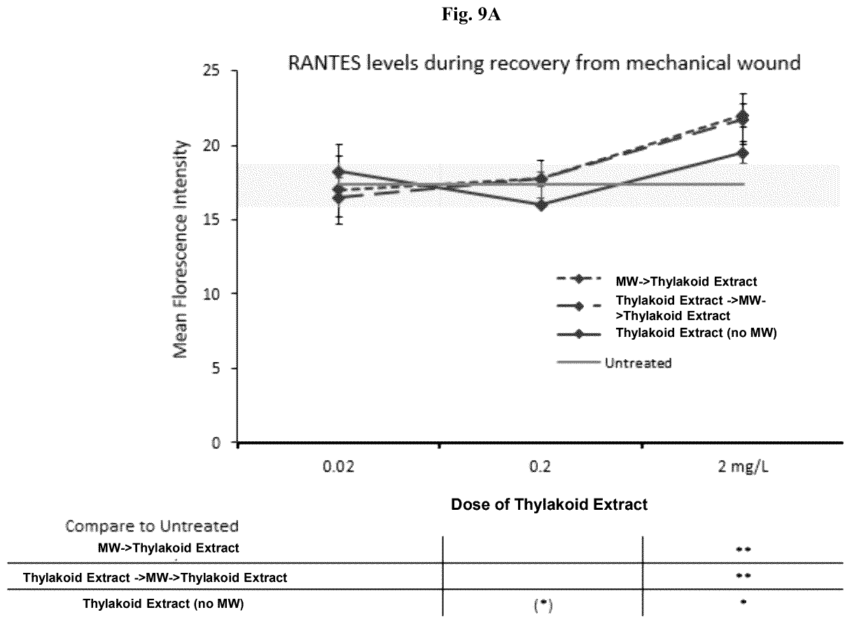

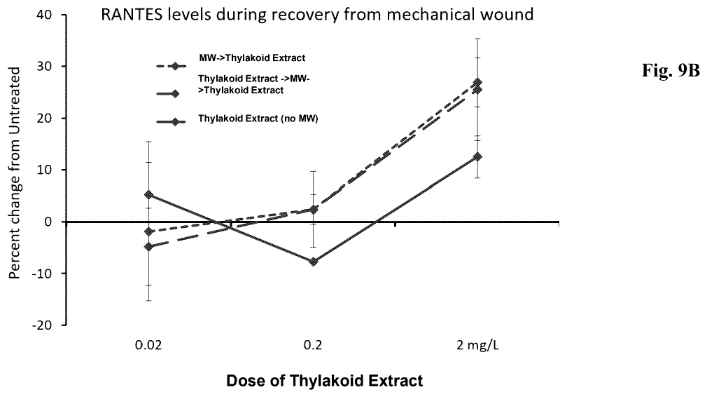

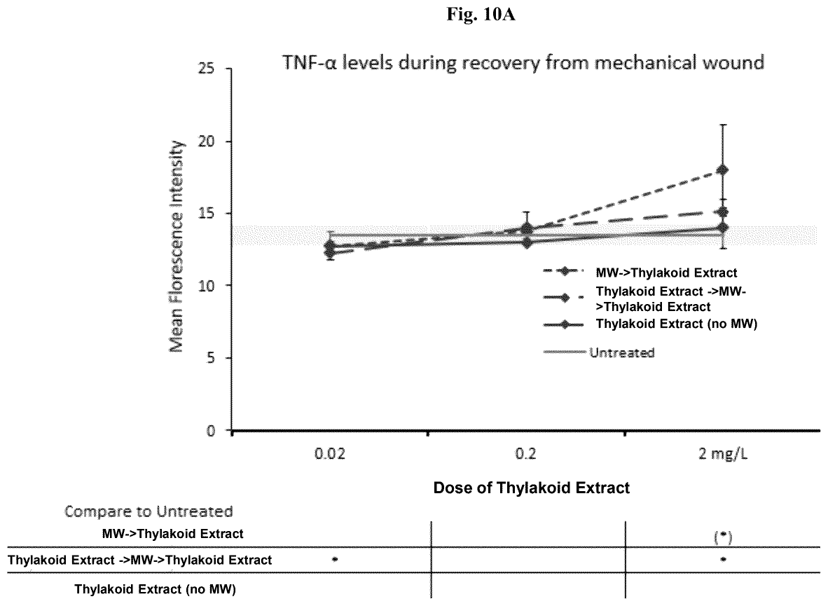

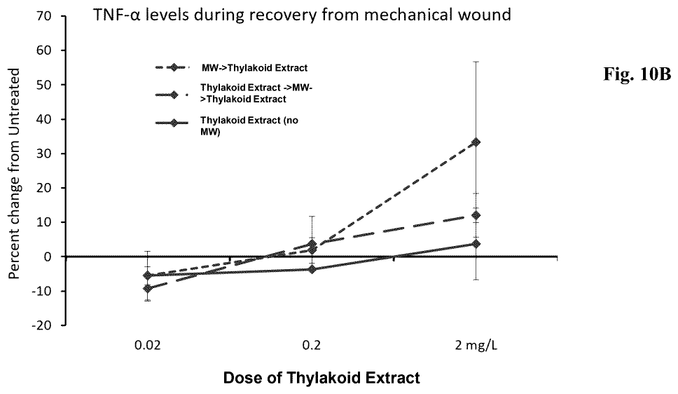

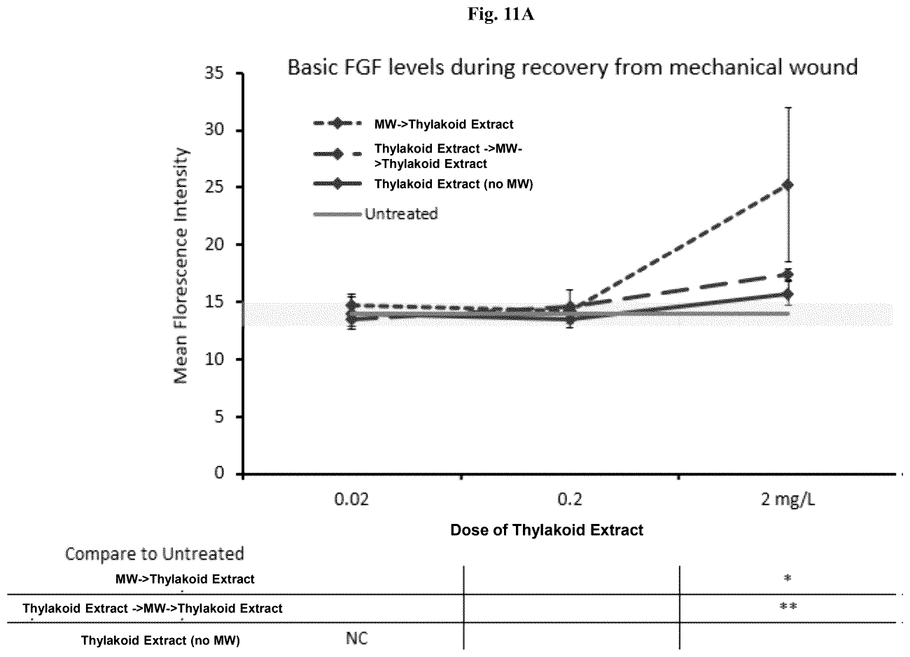

The thylakoid extract was shown to significantly increase the levels of many proinflammatory cytokines, when either given to cells after or before and after the mechanical wound induction, as compared to cells that were untreated or cells treated with thylakoid extract in the absence of a mechanical wound (FIGS. 4A, 4B, 5A, 5B, 6A, 6B, 7A, 7B, 8A, 8B, 9A, 9B, 10A, and 10B). The majority of pro-inflammatory cytokines were increased with the treatment of the thylakoid extract, with the most significant changes were observed by IL-6, IL-8, MCP-1α, and TNF-α (FIGS. 5A, 5B, 6A, 6B, 8A, 8B, 10A, and 10B).

### Proliferative Growth Phase

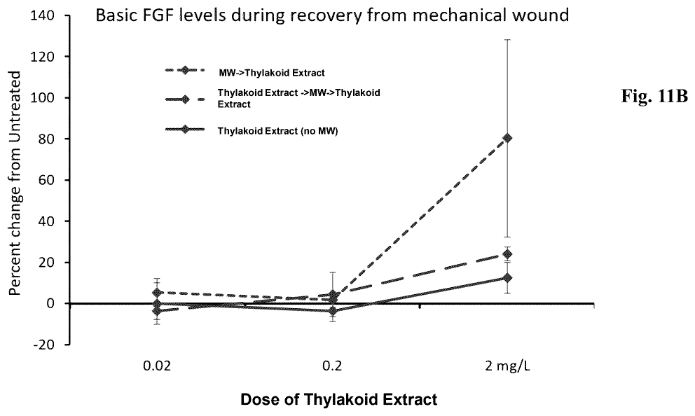

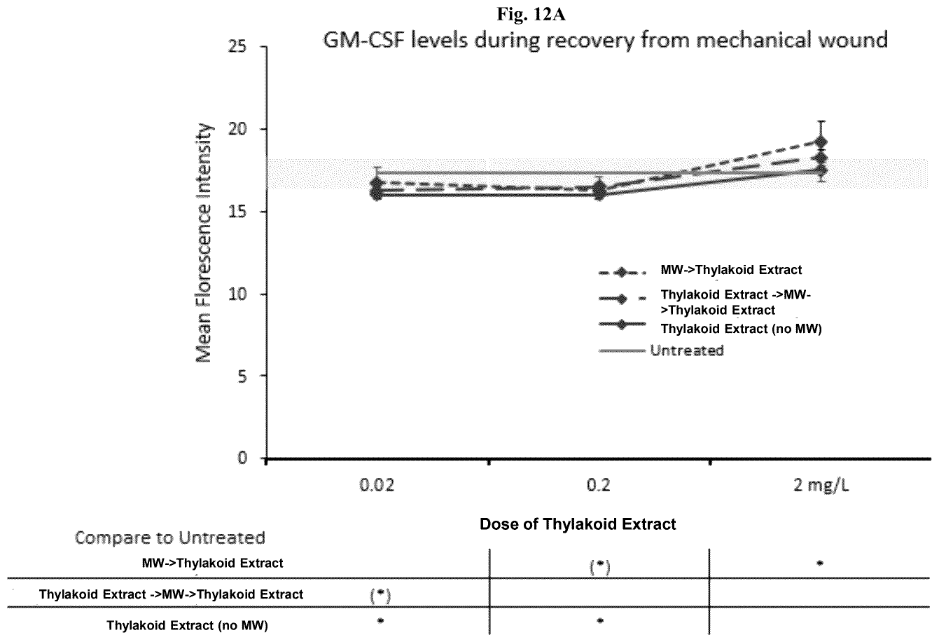

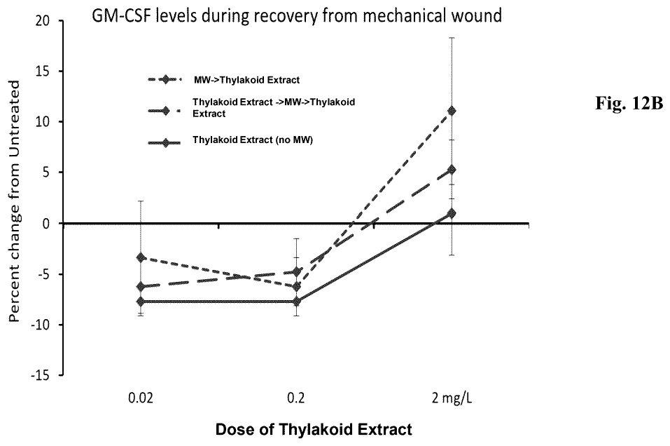

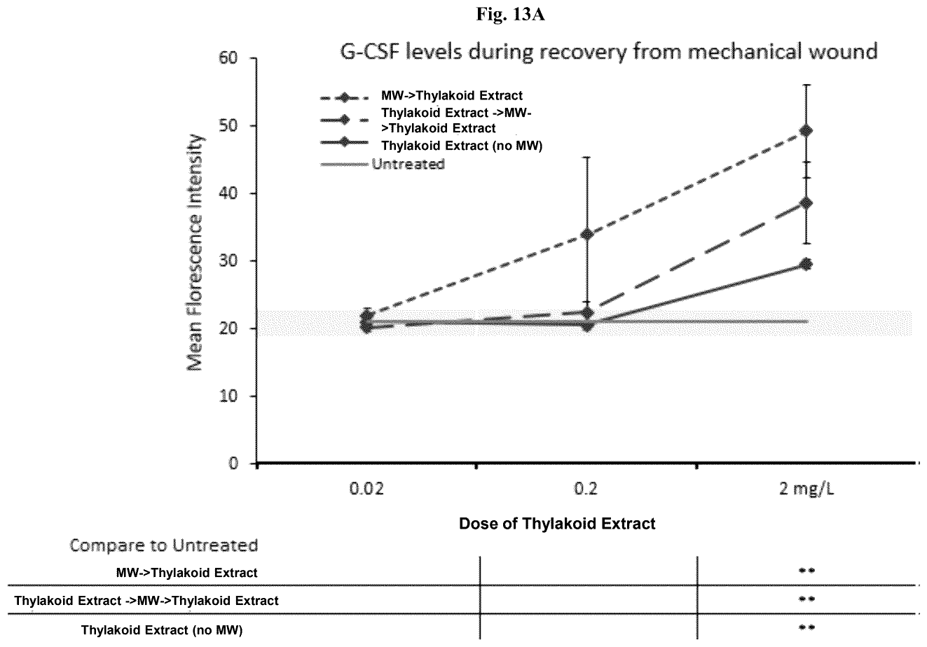

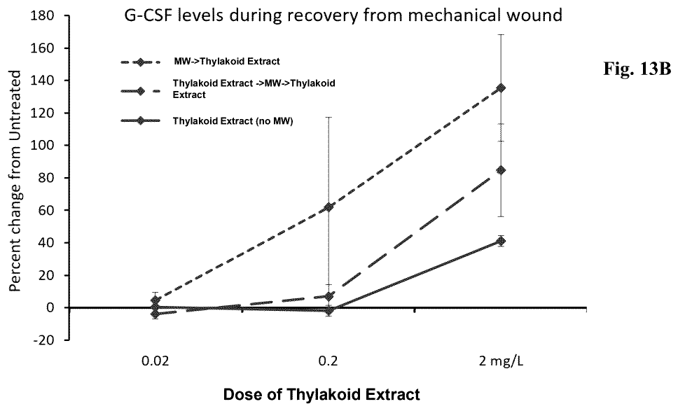

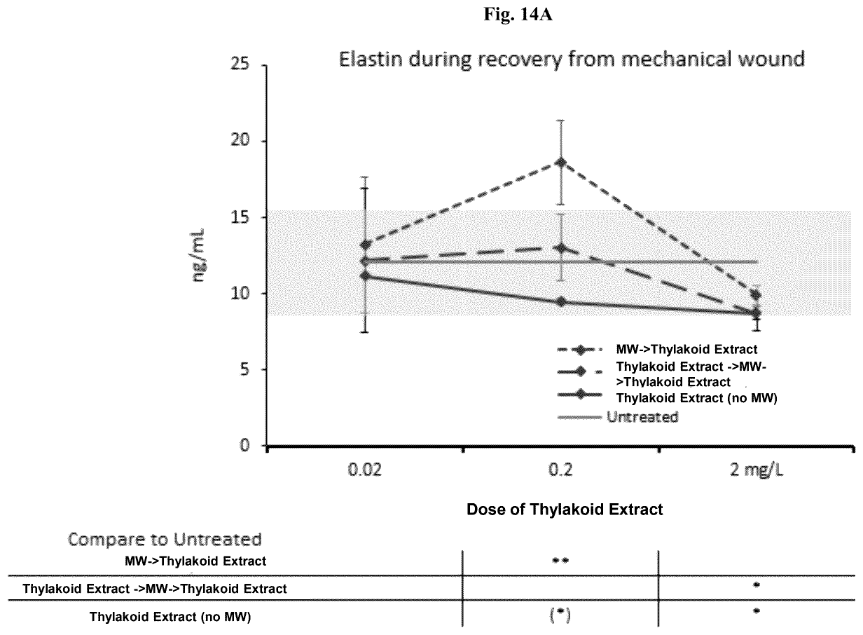

FIGS. 11A, 11B, 12A, 12B, 13A, and 13B show the results of the growth factor multiplex from the in vitro wound healing assay whereby human dermal fibroblasts were treated with the thylakoid extract (0.02 mg/L, 0.2 mg/L, or 2 mg/L) either before or after the mechanical wound. Results are plotted as growth factor (Basic FGF, GM-CSF, or G-CSF) percent change increase or decrease from untreated controls (i.e., cells that were not treated with the thylakoid extract). GM-CSF growth factor is known to play a role in accelerating wound healing and G-CSF growth factor also plays a supportive role in various types of wound healing, including radiation-induced skin damage. G-CSF is further known to help mobilize stem cells and recruit these cells to tissue sites in need of repair. Furthermore, basic FGF triggers fibroblast migration as part of the remodeling phase of wound healing.

The thylakoid extract was shown to significantly increase the levels of three growth factors (Basic FGF, GM-CSF, or G-CSF), when either given to cells after or before and after the mechanical wound induction, as compared to cells that were untreated. Interestingly, increases in Basic FGF, GM-CSF, or G-CSF levels were found to proportional to the thylakoid extract concentration, and thus were highest at the highest concentration of thylakoid extract (2 mg/L) (11A, 11B, 12A, 12B, 13A, and 13B).

### Remodelling Phase

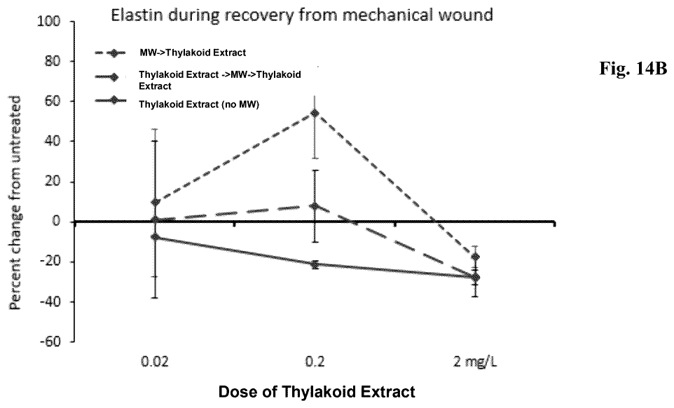

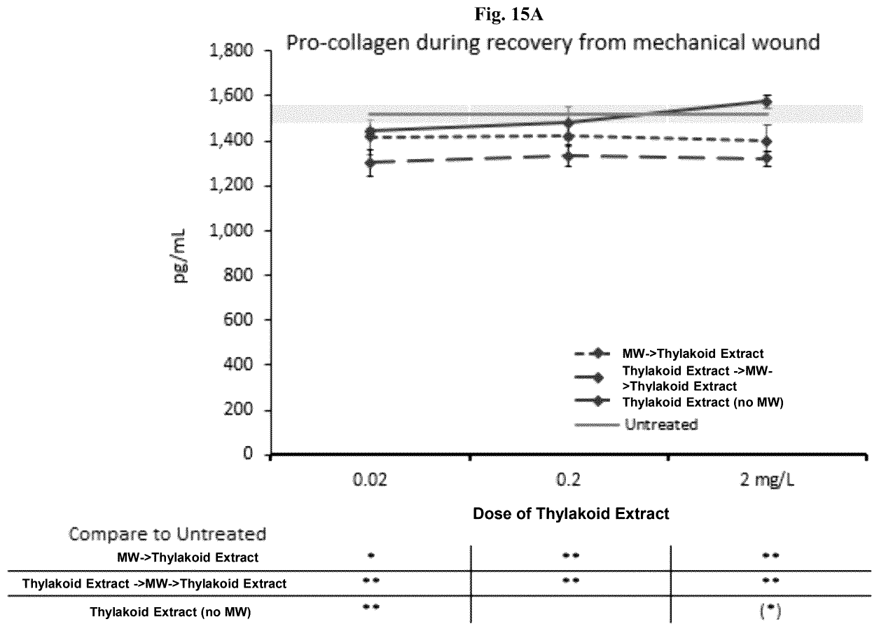

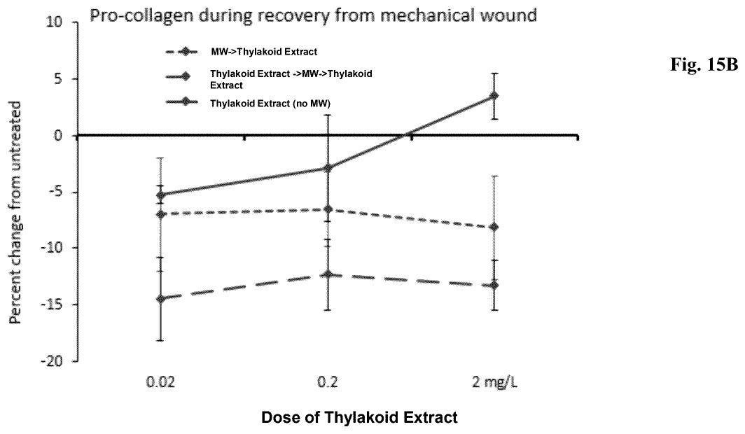

FIGS. 14A, 14B, 15A, and 15B show the results of elastin and pro-collagen (Type I) results from the in vitro wound healing assay whereby human dermal fibroblasts were treated with the thylakoid extract (0.02 mg/L, 0.2 mg/L, or 2 mg/L) either before or after the mechanical wound. Results are plotted as elastin or pro-collagen percent change increase or decrease from untreated controls (i.e., cells that were not treated with the thylakoid extract).

Dermal fibroblasts produce elastin and pro-collagen in culture, and these components help form the extracellular matrix on which the cells adhere, migrate, and maintain an integral cell layer.

The thylakoid extract was shown to significantly decrease levels of pro-collagen, when either given to cells after or before and after the mechanical wound induction, as compared to cells that were untreated, whereas elastin levels were unchanged. The decrease in pro-collagen levels could, however, be attributed to the timing at which the supernatant was assay since pro-collagen is quickly converted to collagen during wound healing.

### CONCLUSION

These data support that the thylakoid extract plays a significant role in each of the natural phases of wound healing. The thylakoid extract increases pro-inflammatory cytokine levels, cell growth factors, and remodeling components to strengthen the skin, reduce scarring, and increase the tensile strength of the skin barrier. These data therefore support the therapeutic effect of thylakoid extract for treating and healing various types of wounds.

### REFERENCES

Vadivel V and Brindha P. Wound Healing Potential of Ipomoea Camea Jacq.: An Un-Explored Herb Used in Indian Traditional 000033 System of Medicine. Glob J Pharmaceu Sci. 2017; 3(1): 555601.
Maxwell (2000): Chlorophyll fluorescence—a practical guide. Journal of Experimental Botany Vol. 51, No. 345, pp. 659-668—April 2000.
Bissonnette E. Y. Proulx L. I., Turmel V., Drouin R., Purcell M. (2004). PCT-233, a novel modulator of pro- and anti-inflammatory cytokine production. Clin. Exp. Immunol., 135: 440-447.

1. A topical composition comprising an effective amount of a photosynthetic cell extract, optionally in admixture with a physiologically acceptable carrier, for increasing GM-CSF growth factor production or levels and/or G-CSF growth factor production or levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

2. A topical composition for use in epithelial (e.g., skin, mucosa, or corneal epithelium) wound healing in a subject, comprising an effective amount of a photosynthetic cell extract, optionally in admixture with a physiologically acceptable carrier, wherein the photosynthetic cell extract is from spinach thylakoid and comprises an active thylakoid extract.

3. The topical composition for use of any of the preceding claims, wherein the extract is present in an amount from about 0.01% to about 0.2% based upon the total weight of the composition.

4. The topical composition for use of any of the preceding claims, wherein the extract is for administration before the wound is present.

5. The topical composition for use of any of the preceding claims, wherein the extract is for administration after the wound is present.

6. The topical composition of any one of the preceding claims, wherein the carrier is selected from the group consisting of fats, hydrophilic or lipophilic gelling agents, solvents, and fillers.

7. The topical composition of any one of the preceding claims wherein the extract is non-lyophilized.

8. The topical composition of any one of the preceding claims, in the form of a solution, a cream, an ointment, an ointment, a foam, a spray, a transdermal patch, or a gel.

9. The topical composition of any one of the preceding claims, wherein the extract is a lyophilized extract reconstituted in water, in physiological saline or any other solution compatible with topical administration, in propylene glycol, or in a solid composition.

10. The topical composition of any one of the preceding claims, wherein the photosynthetic cell extract is from spinach thylakoid and comprises functional thylakoids.

11. The topical composition of any one of the preceding claims, wherein the active thylakoid extract comprises purified functional photosynthetic pigments in their thylakoid membrane environment.

12. The topical composition of any one of the preceding claims, wherein the wound is an open mechanical wound.

13. The topical composition of any one of the preceding claims, wherein the wound comprises skin ulcer (e.g., neuropathic ulcer an ischemic ulcer or a neuroischemic ulcer), an infectious wound, an ischemic wound, a surgical wound, a skin wound from radiation poisoning, or a combination thereof.

14. The topical composition of any one of the preceding claims, wherein the wound is not caused by UVA and/or UVB exposure or by autoimmune disorders or conditions such as eczema.

15. The topical composition of any one of the preceding claims, wherein treatment with the composition increases collagen production or levels in the epithelial tissue, as compared to an untreated control.

16. The topical composition of any one of the preceding claims, wherein treatment with the composition increases elastin production or levels in the epithelial tissue, as compared to an untreated control.

17. The topical composition of any one of the preceding claims, wherein treatment with the composition increases pro-collagen, collagen and/or elastin production or levels in the epithelial tissue, as compared to an untreated control.

18. The topical composition of any one of the preceding claims, wherein treatment with the composition increases basic FGF, G-CSF, and/or GM-CSF production or levels in the epithelial tissue, as compared to an untreated control.

19. The topical composition of any one of the preceding claims, wherein treatment with the composition increases proinflammatory cytokine (e.g., IFN-γ, IL-6, IL-8, IP-10 [CXCL-10], MCP-1, RANTES, and/or TNF-α) production or levels in the epithelial tissue, as compared to an untreated control.

20. Use of a topical composition as defined in any one of the preceding claims for epithelial (e.g., skin, mucosa, or corneal epithelium) wound healing in a subject.

21. Use of a topical composition as defined in any one of the preceding claims for increasing GM-CSF growth factor levels and/or G-CSF growth factor levels in the epithelial tissue (e.g., skin, mucosa, or corneal epithelium) of a subject following an epithelial wound, as compared to an untreated control, wherein the photosynthetic cell extract is from spinach thylakoid and comprises an active thylakoid extract.

22. A method for epithelial (e.g., skin, mucosa, or corneal epithelium) wound healing in a subject in need thereof, said method comprising administering a topical composition as defined in any one of the preceding claims.

23. A method for increasing GM-CSF growth factor levels and/or G-CSF growth factor levels in the epithelial tissue (e.g., skin, mucosa, or corneal epithelium) of a subject following an epithelial wound, as compared to an untreated control, said method comprising administering a topical composition as defined in any one of the preceding claims.
